(19) United States
(12) Patent Application Publication
(10) Pub. No.: US 2009/0088728 A1
(43) Pub. Date: Apr. 2, 2009

(54) MALLEABLE SLEEVE FOR BALLOON CATHETER AND ENDOSCOPIC SURGICAL METHOD

(76) Inventors: Michael L. Dollar, Garland, TX (US); Victor A. Dubuclet, IV, Dallas, TX (US)

Correspondence Address: CARSTENS & CAHOON, LLP, P O BOX 802334, DALLAS, TX, 75380, US

(21) Appl. No.: 11/864,124

(22) Filed: Sep. 28, 2007

(51) Int. Cl. A61M 25/01

(52) U.S. Cl. 604/528

(57) ABSTRACT

The present invention is directed towards a malleable sleeve used in conjunction with a multi-lumen catheter with an expandable device, such as a balloon, incorporated into its surface. The malleable sleeve allows the catheter to be pre-formed into a shape that corresponds with the shape of the nasal passageway. The present invention also covers endoscopic surgical methods utilizing the malleable sleeve.

### FIELD OF THE INVENTION

This invention relates to surgical catheters and methods for using such catheters for treating paranasal airways.

### BACKGROUND OF THE INVENTION

In order to fully understand this invention, it is important to consider the anatomy of the sinus system. The sinus system consists of many different pathways, called ducts or ostia, which allow mucus, air and other substances to drain and flow through the system. Inflammation can occur in the tissues that make up the ducts and ostia, causing them to swell and block the normal flow. Inflammation may be caused by allergies, noxious agents, nasal polyps, and other factors. Over time there can be a pathologic increase in inflamed tissue causing permanent disruption in the flow through the sinus system. Obstruction of the narrow ducts and ostia between the paranasal sinuses and nasal cavity develops, resulting in a vicious cycle of increased secretions, edema and ultimately complete blockage of the sinus pathways. The state of chronic sinus inflammation is called sinusitis.

Treatment with antibiotics, antihistamines, and corticosteroids in nasal sprays or systematically may result in effective resolution of sinusitis. However, some patients become resistant to medical treatment and surgery becomes necessary. Endoscopic sinus surgery is performed from an intranasal approach, thus eliminating the need for external incisions. A minimally invasive type of endoscopic surgery, called balloon catheterization or sinuplasty, involves placing an expandable device, such as a deflated balloon, inside the clogged sinus pathways and inflating the balloon in order to open the clogged pathway. This type of surgery has also been used to open clogged pathways in other body systems, including in the vascular system, the urinary tract, and the lacrimal system. Some catheters are “steerable catheters” in that they incorporate a means that allows surgeons to deflect the tip in at least one direction, thereby allowing the surgeon to “steer” the tip of the catheter to the region of interest inside the body. Steerable catheters typically contain one or two small lumens that run the length of the catheter and house steering wires. The steering wires are attached to the tip of the catheter so that pulling on one of the steeling wires will deflect the tip in the direction of the steering wire. The present invention will work with either the “steerable” type of catheter or the “non-steerable” type of catheter.

### SUMMARY OF THE INVENTION

The present invention is thus directed towards a malleable sleeve for a surgical catheter and endoscopic method of surgery utilizing the malleable sleeve to perform balloon catheterization.

The first embodiment of the present invention is a malleable sleeve designed to be used with any flexible balloon catheter. The malleable sleeve slides over the balloon catheter shaft and is fixed in place far enough away from the tip of the balloon catheter to allow the balloon to be inflate. The malleable sleeve is soft enough to allow a surgeon to bend it by hand, but hard enough to retain its shape once the surgeon has bent it. The surgeon is able to bend the malleable sleeve into the shape of the specific sinus or other passageway through which the catheter will be traveling. This reduces the pressure on the nasal passages that typically occurs during endoscopic balloon catheter surgery. It also allows the surgeon to customize the catheter shape to each individual patient's unique anatomy.

The endoscopic surgical method utilizing the first embodiment of the present invention involves using the malleable sleeve to pre-form the catheter shaft to the shape of the particular sinus pathway traveled by the balloon catheter. The surgeon first determines the path to the region inside the patient's sinus system causing sinusitis. The surgeon then bends the malleable sleeve into a shape consistent with the path that will be followed by the balloon catheter. The surgeon slides the malleable sleeve over a balloon catheter, inserts the balloon catheter into the patient's nasal cavity, and guides it to the affected region. Finally, the surgeon inflates the balloon for a predetermined period of time, deflates the balloon, and removes the catheter.

The second embodiment of the present invention is intended to be used with a multi-lumen catheter. The multi-lumen catheter used in conjunction with the second preferred embodiment has an expandable device, such as a balloon, built into its surface near the tip. One of the lumens inside the multi-lumen catheter terminates at the surface of the catheter shaft inside the balloon and is used to inflate the balloon. Another lumen contains an opening to the surface of the catheter shaft near the balloon. The malleable sleeve has a flange built into its surface biased towards the central axis of the malleable sleeve. In its initial position, the flange is maintained approximately radially coterminous with the malleable sleeve shaft. In its secondary position, the bias of the flange causes it to bend down into the opening in the multi-lumen catheter shaft. The flange then acts as a ramp, allowing the surgeon to easily move an imaging instrument through the lumen, up the ramp, and out the opening, giving the surgeon a clear view of the balloon built into the surface of the multi-lumen catheter.

The endoscopic surgical method utilizing the second embodiment of the present invention uses both the malleable sleeve element and the flange element to accomplish quick and effective relief of chronic sinusitis. As with the method utilizing the first embodiment, the surgeon views the pathway through which the balloon catheter will travel, and bends the malleable sleeve over the multi-lumen catheter corresponding to the shape of the sinus pathways. Before the surgery begins, the malleable sleeve is in its initial position relative to the multi-lumen catheter shaft. The surgeon then inserts the malleable sleeve and multi-lumen catheter into the sinus pathway. Once the tip of the multi-lumen catheter reaches the obstructed pathway, the surgeon can perform a traditional balloon sinuplasty through one of the lumens in the multi-lumen catheter shaft. Next, the surgeon moves the balloon tip of the multi-lumen catheter into the obstructed pathway. The surgeon then partially retracts the imaging device to a point behind the opening in the multi-lumen catheter shaft near the balloon. The surgeon moves the malleable sleeve into its secondary position causing the flange to bend down into the multi-lumen catheter shaft opening. The surgeon then slides the imaging device up the ramp created by the flange and out the catheter shaft, giving the surgeon a clear view of the balloon built into the tip of the multi-lumen catheter shaft. The surgeon then inflates the balloon for a predetermined amount of time, deflates the balloon, retracts the imaging device, and retracts the multi-lumen catheter and malleable sleeve.

The third and fourth embodiment of the present invention is a malleable sleeve with two lumens. This malleable sleeve slides over a balloon catheter through one lumen just like the first two embodiments. These embodiments differ from the first two embodiments in that they provide a second lumen that carries an imaging device. Sliding an imaging device through the second lumen of the malleable sleeve allows a surgeon to get a clear view of the inflation of the balloon at the tip of the balloon catheter.

The endoscopic surgical method utilizing the third or fourth embodiment of the present invention differs from the first two methods only in the way the imaging device is positioned behind the balloon. Instead of sliding the imaging device down one of the lumens in the multi-lumen catheter, the imaging device is slid through the second lumen in the multi-lumen malleable sleeve.

### BRIEF DESCRIPTION OF THE DRAWINGS

A more complete understanding of the method of the present invention may be had by reference to the following detailed description when taken in conjunction with the accompanying drawings, wherein:

FIG. 1 is an angled view of the first embodiment of the malleable sleeve of the present invention before it is bent, and a steerable multi-lumen catheter;

FIG. 2 is an angled view of the malleable sleeve after it has been bent, and a steerable multi-lumen catheter;

FIG. 3A is a cross-sectional view of a steerable balloon catheter inside the nasal cavity in the process of widening an obstructed pathway without the malleable sleeve of the present invention;

FIG. 3B is a cross-sectional view of the malleable sleeve of the present invention being used in conjunction with a steerable balloon catheter inside the nasal cavity in the process of widening an obstructed pathway;

FIG. 4 is an angled outside view of the malleable sleeve of the second embodiment of the present invention depicting the flanged section of the malleable sleeve shaft, and a steerable multi-lumen catheter with a balloon built into its surface and a lumen opening at its surface near the balloon;

FIG. 5A is a cross-sectional view of the second embodiment of the malleable sleeve of the present invention in its initial position over the multi-lumen catheter shaft;

FIG. 5B is a cross-sectional view of the second embodiment of the malleable sleeve of the present invention in its secondary position over the multi-lumen catheter shaft;

FIG. 6 is an angled view of the third embodiment of the present invention and a steerable balloon catheter;

FIG. 7 is an angled view of the fourth embodiment of the present invention and a steerable balloon catheter;

FIG. 8 is a flowchart depicting the surgical method that utilizes the first embodiment of the present invention;

FIG. 9 is a flowchart depicting the surgical method that utilizes the second embodiment of the present invention;

FIG. 10 is a flowchart depicting the surgical method that utilizes the third or fourth embodiment of the present invention;

Where used in the various figures of the drawing, the same numerals designate the same or similar parts. Furthermore, when the terns “top,” “bottom,” “first,” “second,” “upper,” “lower,” “height,” “width,” “length,” “end,” “side,” “horizontal,” “vertical,[ and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

The present invention is directed towards a malleable sleeve for a balloon catheter and method for using it to perform endoscopic surgery.

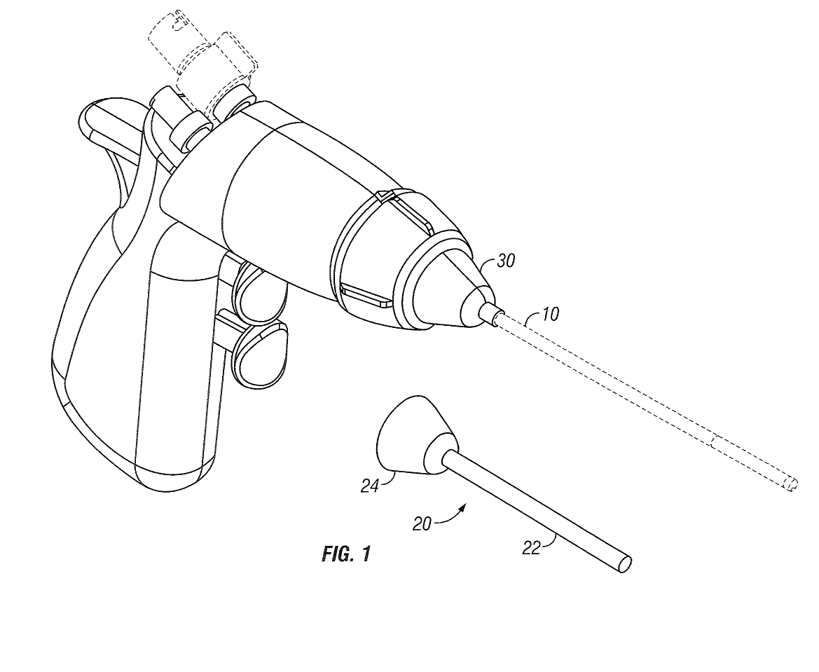

Referring initially to FIG. 1, therein is depicted a steerable catheter and the first embodiment of the present invention. The malleable sleeve 20 is depicted therein in its non-deformed state prior to surgery. The malleable sleeve has a malleable shaft element 22, and a coupling element 24 adapted to mate with the steerable catheter 10 at its base 30. Although the embodiment shown depicts a coupling element conical in shape, it is understood that the present invention contemplates coupling elements adapted to fit catheter bases of varying sizes and shapes. The malleable shaft element 22 is made of any malleable material such as a plastic, metal, or a combination thereof having physical properties that allow the shaft to be bent by hand and retain its shape.

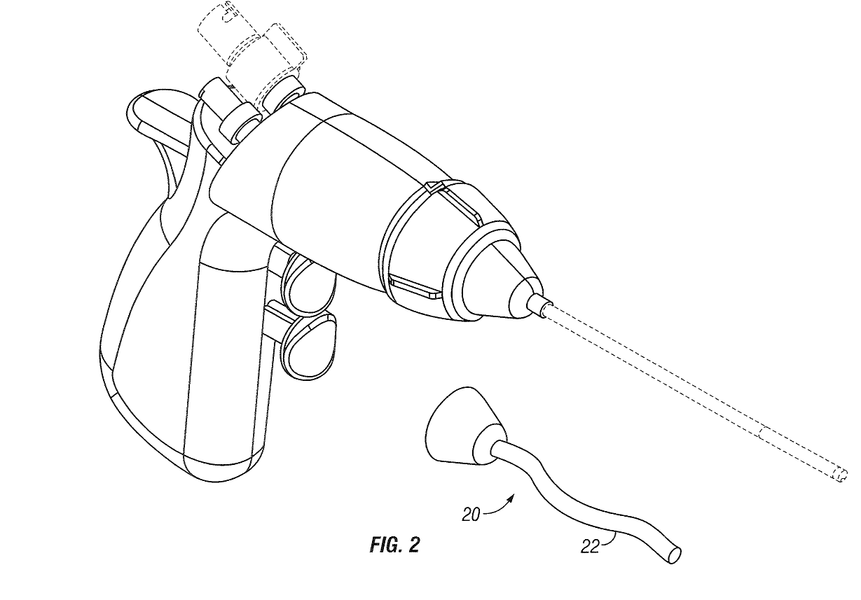

Referring now to FIG. 2, therein is depicted the malleable sleeve 20 after it has been bent into a shape corresponding to the nasal passageway through which the catheter will travel. The shape depicted in FIG. 2 is only for illustrative purposes and is not intended to limit the scope of the present invention. The shape of the shaft 22 of the malleable sleeve 20 is determined by the judgment of the surgeon and the individual anatomy of the patient.

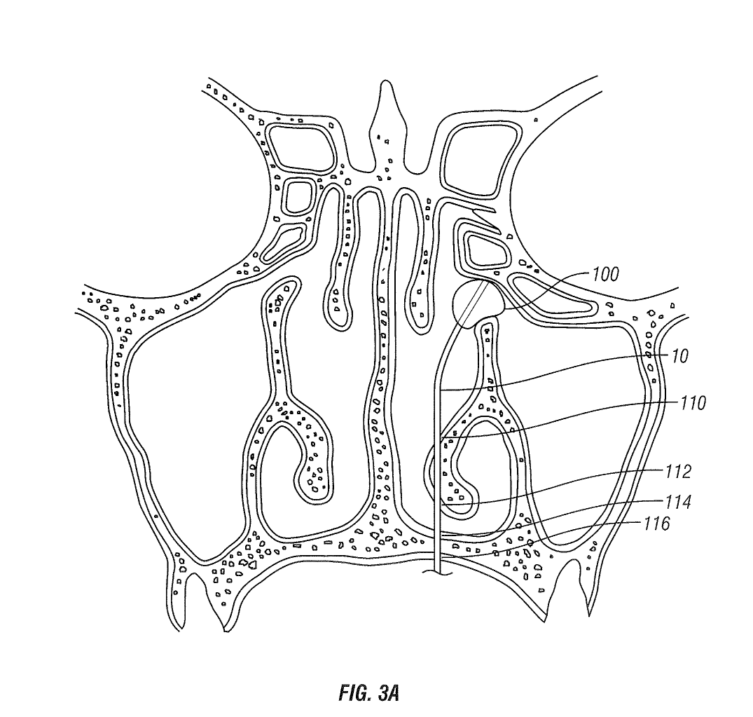

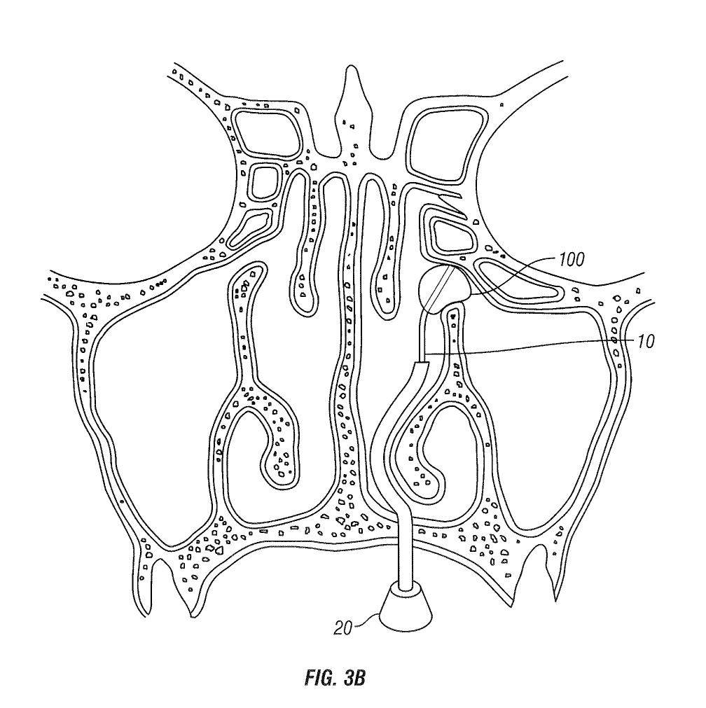

Referring next to FIG. 3A, therein is depicted a balloon catheter 10 without the malleable sleeve of the present invention inside the nasal cavity in the process of enlarging an opening 100. It can be seen from the figure that a balloon catheter 10 without the malleable sleeve of the present invention will put pressure on several different locations 110, 112, 114, and 116 inside the nasal cavity. It is also more difficult to navigate the nasal cavity passageways without the assistance of the malleable sleeve.

Referring next to FIG. 3B, therein is depicted a balloon catheter 10 inserted into the nasal cavity using the assistance of the malleable sleeve 20 of the present invention. As can be seen in the figure, the anatomy of the sinus can be navigated more easily and more comfortably for the patient using the malleable sleeve 20 as a guide.

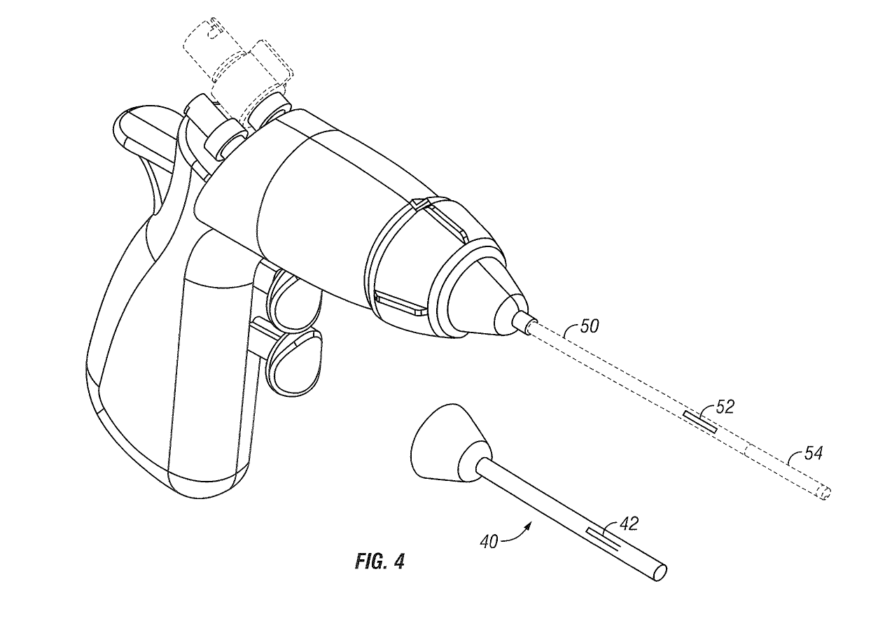

Referring next to FIG. 4, therein is depicted the second embodiment of the present invention. This embodiment of the malleable sleeve 40 has a flange 42 built into its surface that is biased towards the axis of the sleeve. This embodiment is used in conjunction with a multi-lumen catheter 50 that has an opening in its shaft 52 that approximately corresponds to the location of the flange 42 on the malleable sleeve 40. Also, as seen in FIG. 4, the length of the malleable sleeve 40 must be such that it is able to slide over the multi-lumen catheter 50 far enough that the malleable sleeve does not interfere with the inflation of the balloon 54 built into the tip of the multi-lumen catheter.

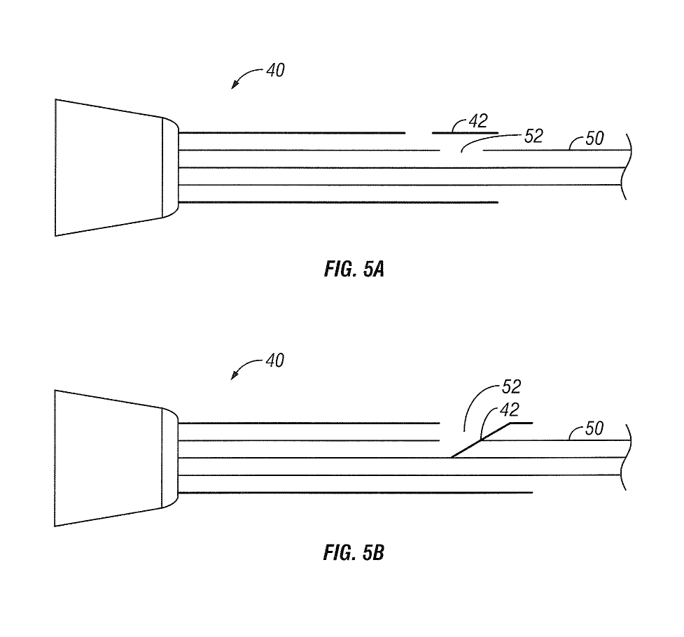

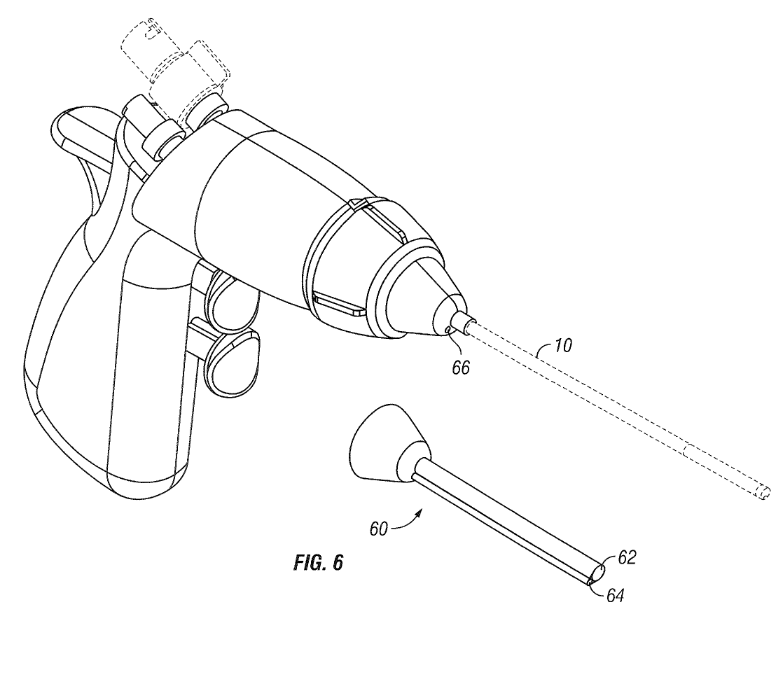

Referring next to FIG. 5A, therein is depicted a cross section of the malleable sleeve 40 of the present invention in its initial position over the multi-lumen catheter 50. The initial position of the malleable sleeve 40 is a position such that the shaft of the multi-lumen catheter 50 supports the flange 42 built into the wall of the malleable sleeve. The wall of the multi-lumen catheter keeps the flange 42 approximately radially coterminous with the wall of the malleable sleeve (ie. keeps the flange from bending towards the central axis of the malleable sleeve).

The secondary position of the malleable sleeve is depicted in FIG. 5B. When the malleable sleeve 40 is in the secondary position, the flange 42 is allowed to bend towards the central axis of the malleable sleeve and into the opening 52 in the shaft of the multi-lumen catheter 50. The flange 42 creates a type of inclined ramp that helps an imaging device slide through the lumen and easily slide out the opening 52 in the multi-lumen catheter shaft 50. As used herein, in either the specification or the claims, the term “imaging device” is defined as including fiberscopes, endoscopes and all other long, thin, flexible viewing instruments used by surgeons and those skilled in the art to view the interior of the human body. The second embodiment of the present invention can optionally incorporate a locking means (not shown), such as a series of grooves and threads, adapted to temporarily holding the malleable sleeve in its initial and secondary positions, depending on the needs of the surgeon.

Referring next to FIG. 6, therein is depicted the third embodiment of the present invention. This malleable sleeve 60 has a primary lumen 62 large enough to allow it to slide over a balloon catheter 10 to a point that it does not interfere with inflation of the balloon. In addition, it has a second, smaller lumen 64 meant to carry an imaging device. The second lumen 64 runs the length of the malleable sleeve 60 and is open at the tip of the malleable sleeve. An imaging device can be slid through the entry hole 66 for the second lumen 64, down the length of the second lumen and out the opening at the tip, giving the surgeon a clear view of the balloon at the tip of the catheter 10.

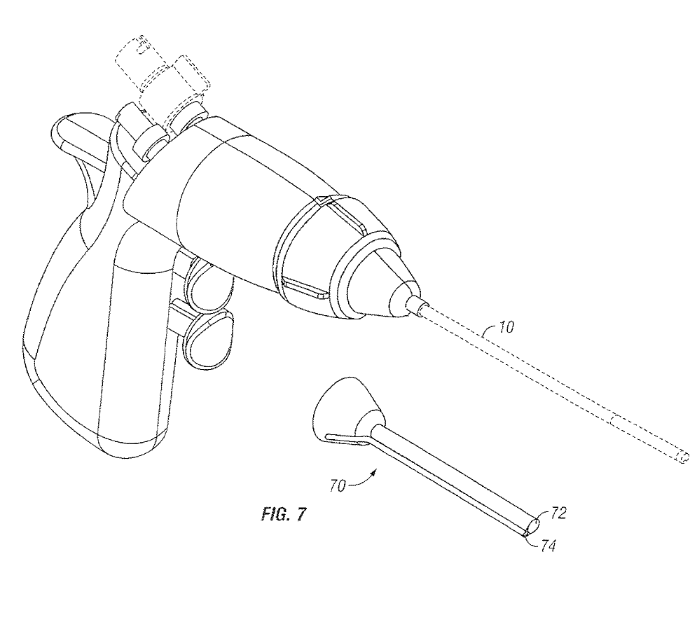

Referring next to FIG. 7, therein is depicted the fourth embodiment of the present invention. This malleable sleeve 70 has a primary lumen 72 large enough to allow it to slide over a balloon catheter 10 to a point that it does not interfere with inflation of the balloon. In addition, it has a second, smaller lumen 74 meant to carry an imaging device. The second lumen 74 runs the length of the malleable sleeve 70 and is open at the tip of the malleable sleeve. An imaging device can be slid down the length of the second lumen 74 and out the opening at the tip, giving the surgeon a clear view of the balloon at the tip of the catheter 10.

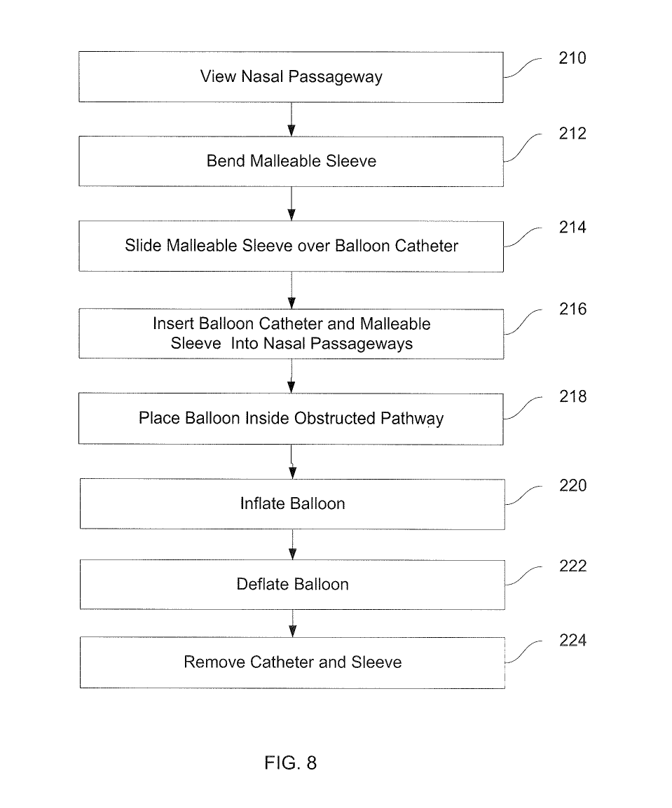

The endoscopic surgical method utilizing the first embodiment of the present invention is depicted in the flow chart in FIG. 8. First, the surgeon uses an imaging device, MRI, CT or other image guidance means to view the nasal passageway 210 and determine the path to the region inside the patient's body causing sinusitis. The surgeon uses this information to bend the malleable sleeve 212 into a shape consistent with the path that will be followed by the balloon catheter. Next, the surgeon slides the malleable sleeve over a balloon catheter 214, inserts the balloon catheter into the patient's nasal cavity 216 and guides it into the affected region 218. The surgeon verifies placement of the balloon using an imaging device. Finally, the surgeon inflates the balloon 220 for a predetermined period of time, deflates the balloon 222, and removes the catheter and malleable sleeve 224 from the sinus system.

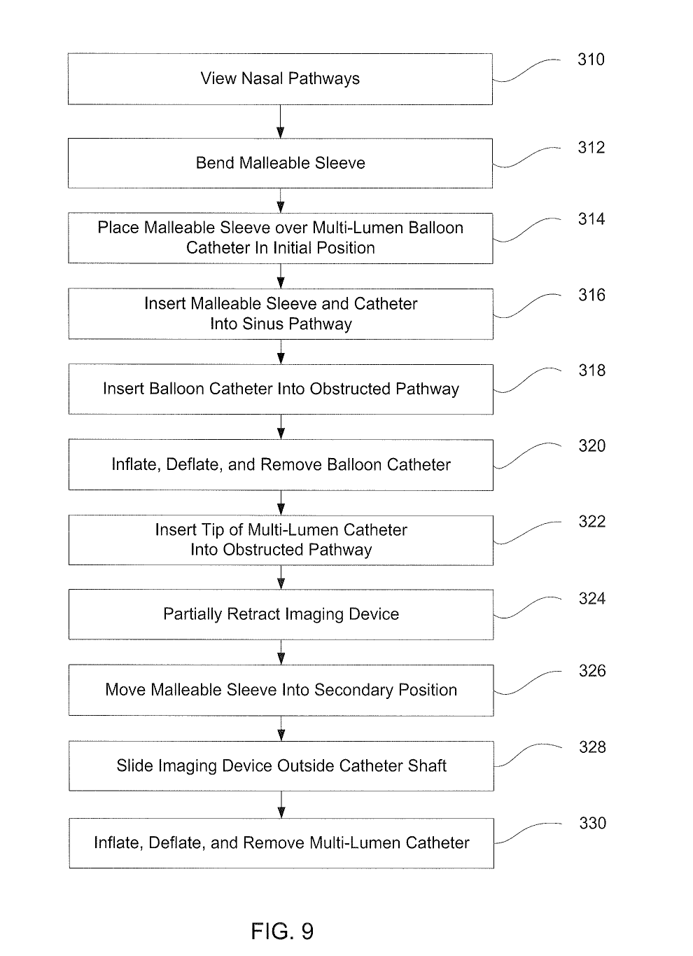

The endoscopic surgical method utilizing the second embodiment of the present invention is depicted in FIG. 9. First, the surgeon views the pathway through which the balloon catheter will travel 310. The surgeon then bends the malleable sleeve 312 into the appropriate shape determined using the information obtained from the initial exploration of the sinus cavity. The surgeon places the bent malleable sleeve over a multi-lumen catheter 314 with a balloon built into its surface at the tip. The multi-lumen catheter must also have at least one lumen with two openings: one opening at the tip of the multi-lumen catheter and one opening in the shaft of the multi-lumen catheter shaft near the proximal end of the balloon. The surgeon places the malleable sleeve in its initial position relative to the opening in the multi-lumen catheter shaft 314. Next, the surgeon inserts an imaging instrument into the lumen containing the opening in the shaft of the multi-lumen catheter. At this stage, however, the imaging instrument runs past the shaft opening, all the way to the tip of the multi-lumen catheter. The surgeon then inserts the malleable sleeve and multi-lumen catheter into the sinus pathway 316. Once the tip of the multi-lumen catheter shaft reaches the obstructed pathway, the surgeon can perform a traditional balloon catheter surgery through one of the lumens in the multi-lumen catheter shaft 318 and 320. Next, the surgeon moves the balloon tip of the multi-lumen catheter into the obstructed pathway 322. The surgeon then partially retracts the imaging device 324 to a point behind the opening in the multi-lumen catheter shaft. The surgeon moves the malleable sleeve into its secondary position 326 causing the flange to bend down into the multi-lumen catheter shaft opening. The surgeon then slides the imaging device up the ramp created by the flange and out the catheter shaft 328, giving the surgeon a clear view of the balloon built into the tip of the multi-lumen catheter shaft. The surgeon then inflates the balloon for a predetermined amount of time, deflates the balloon, retracts the imaging device, and retracts the multi-lumen catheter and malleable sleeve 330.

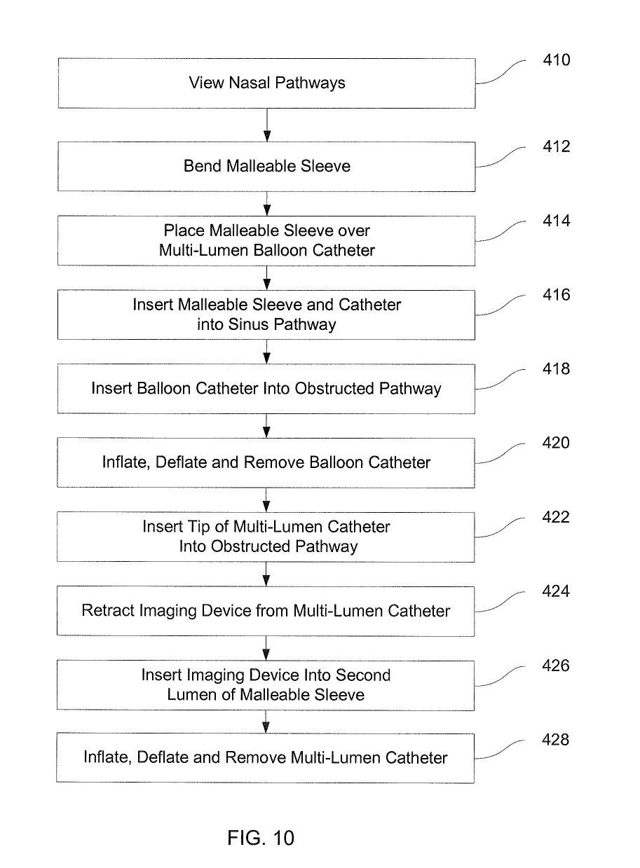

The endoscopic surgical method utilizing the third or fourth embodiment of the present invention is depicted in FIG. 10. First, the surgeon views the pathway through which the balloon catheter will travel 410. The surgeon then bends the malleable sleeve into the appropriate shape 412, determined using the information obtained from the initial exploration of the sinus cavity. The surgeon the places the malleable sleeve over a multi-lumen catheter shaft 414. The multi-lumen catheter must have a balloon built into its surface at the tip. The multi-lumen catheter must also have at least three lumens: one dedicated to inflating and deflating the balloon, and two that are open at the tip of the catheter and can be used to insert imaging devices, balloon catheters, or other surgical instruments into the nasal cavity. Next, the surgeon inserts an imaging device into one of the lumens of the multi-lumen catheter. The surgeon then inserts the malleable sleeve and multi-lumen catheter shaft through the sinus pathway 416 and into the obstructed pathway 418. Once the tip of the multi-lumen catheter shaft reaches the obstructed pathway, the surgeon can perform a traditional balloon sinuplasty surgery through one of the lumens in the multi-lumen catheter shaft 420. Next, the surgeon moves the balloon tip of the multi-lumen catheter into the obstructed pathway 422. The surgeon then slides the imaging device out of the multi-lumen catheter 424, and inserts the imaging device into the second lumen of the malleable sleeve until it reaches the opening at the distal end of the malleable sleeve 426. Once the surgeon has a clear view of the balloon in the obstructed pathway, the balloon can be inflated and deflated to open the obstructed nasal pathway 428.

We claim:

1. A malleable sleeve for a balloon catheter comprising:
(a) a tubular malleable shaft defining a first lumen with a proximal end and a distal end;
(b) a base connected to the proximal end of the malleable shaft and adapted to mate with a base of a multi-lumen catheter;

2. The malleable sleeve of claim 1 additionally comprising a flange built into the malleable shaft biased towards the central axis of the malleable shaft.

3. The malleable sleeve of claim 1 additionally comprising a second lumen approximately equal in length to the first lumen.

4. An endoscopic surgical method, comprising the steps of:
(a) providing a human body with a nasal system having at least one obstructed fluid pathway;
(b) providing a balloon catheter and an imaging device;
(c) providing a multi-lumen catheter with a surface, a base, a tip, an expandable device incorporated into its surface near the tip and a first, second, and third lumen, with the first lumen adapted to expanding and contracting the expandable device;
(d) providing a malleable sleeve for the multi-lumen catheter having a base adapted to mate with the base of the multi-lumen catheter;
(e) viewing the nasal system;
(f) bending the malleable sleeve into a shape approximating the nasal system;
(g) sliding the malleable sleeve over the multi-lumen catheter until the malleable sleeve base mates with the base of the multi-lumen catheter;
(h) inserting the imaging device in the third lumen of the multi-lumen catheter;
(i) inserting the malleable sleeve and multi-lumen catheter into the nasal system;
(j) guiding the multi-lumen catheter tip to the obstructed fluid pathway;
(k) inserting the balloon catheter into the obstructed fluid pathway through the second lumen;
(l) inflating the balloon catheter;
(m) deflating the balloon catheter;
(n) removing the balloon catheter from the nasal system through the second lumen;
(o) inserting the expandable device incorporated into the surface of the multi-lumen catheter into the obstructed fluid pathway;
(p) expanding the expandable device;
(q) contracting the expandable device; and
(r) removing the multi-lumen catheter and malleable sleeve from the nasal system.

5. The endoscopic surgical method of claim 4 comprising the additional step of repeating steps (p) and (q) at least once.

6. An endoscopic surgical method, comprising the steps of:
(a) providing a human body with a nasal system having at least one obstructed fluid pathway;
(b) providing a balloon catheter and an imaging device;
(c) providing a multi-lumen catheter with a surface, a base, a tip, an expandable device incorporated into its surface near the tip having a proximal end and a distal end, a first lumen, a second lumen, and third lumen, with the first lumen adapted to expanding and contracting the expandable device, and the third lumen having two openings, a first opening at the tip of the multi-lumen catheter, and a second opening in the shaft of the multi-lumen catheter near the proximal end of the expandable device;
(d) providing a malleable sleeve with a base adapted to made with the base of the multi-lumen catheter and a flange biased towards the central axis of the malleable sleeve and located approximately corresponding to the location of the second opening in the third lumen of the multi-lumen catheter;
(e) viewing the nasal system;
(f) bending the malleable sleeve into a shape approximating the nasal system;
(g) sliding the malleable sleeve over the multi-lumen catheter until the malleable sleeve base mates with the base of the multi-lumen catheter in an initial position;
(h) inserting the imaging device in the third lumen of the multi-lumen catheter;
(i) inserting the malleable sleeve and multi-lumen catheter into the nasal system;
(j) guiding the multi-lumen catheter tip to the obstructed fluid pathway;
(k) inserting the balloon catheter into the obstructed fluid pathway through the second lumen;
(l) inflating the balloon catheter;
(m) deflating the balloon catheter;
(n) removing the balloon catheter from the nasal system through the second lumen;
(o) inserting the expandable device incorporated into the surface of the multi-lumen catheter into the obstructed fluid pathway;
(p) retracting the imaging device to a point in the third lumen behind the second opening;
(q) moving the malleable sleeve into a secondary position thereby allowing the flange to bend towards the central axis of the multi-lumen catheter and into the second opening of the third lumen;
(r) sliding the imaging device past the flange and through the second opening in the third lumen of the multi-lumen catheter.
(s) expanding the expandable device;
(t) contracting the expandable device;
(u) retracting the imaging device into the third lumen of the multi-lumen catheter; and
(v) removing the multi-lumen catheter and malleable sleeve from the nasal system.

7. The endoscopic surgical method of claim 6 comprising the additional step of repeating steps (s) and (t) at least once.

8. An endoscopic surgical method, comprising the steps of:
(a) providing a human body with a nasal system having at least one obstructed fluid pathway;
(b) providing a balloon catheter and an imaging device;
(c) providing a multi-lumen catheter with a surface, a base, a tip, an expandable device incorporated into its surface near the tip having a proximal end and a distal end, a first lumen, a second lumen, and third lumen, with the first lumen adapted to expanding and contracting the expandable device;
(d) providing a malleable sleeve with at least two lumens, a first lumen with an inside diameter large enough to allow the multi-lumen catheter to pass through it, and a second lumen with an inside diameter large enough to allow the imaging device to pass through it;
(e) viewing the nasal system;
(f) bending the malleable sleeve into a shape approximating the nasal system;
(g) sliding the malleable sleeve over the multi-lumen catheter until the malleable sleeve base mates with the base of the multi-lumen catheter;
(h) inserting the imaging device in the third lumen of the multi-lumen catheter;
(i) inserting the malleable sleeve and multi-lumen catheter into the nasal system;
(j) guiding the catheter tip to the obstructed fluid pathway;
(k) inserting the balloon catheter into the obstructed fluid pathway through the second lumen;
(l) inflating the balloon catheter;
(m) deflating the balloon catheter;
(n) removing the balloon catheter from the nasal system through the second lumen;
(o) inserting the expandable device incorporated into the surface of the multi-lumen catheter into the obstructed fluid pathway;
(p) retracting the imaging device out of the third lumen;
(q) inserting the imaging device into the second lumen of the malleable sleeve;
(r) sliding the imaging device down the second lumen of the malleable sleeve until a view of the expandable device is obtained.
(s) expanding the expandable device;
(t) contracting the expandable device;
(u) retracting the imaging device out of the second lumen of the malleable sleeve; and
(v) removing the multi-lumen catheter and malleable sleeve from the nasal system.

9. The endoscopic surgical method of claim 8 comprising the additional step of repeating steps (s) and (t) at least once.
